(19) United States
(12) Patent Application Publication
(10) Pub. No.: US 2022/0249015 A1
(43) Pub. Date: Aug. 11, 2022

(54) METHOD FOR NEAR REAL-TIME SLEEP DETECTION IN A WEARABLE DEVICE BASED ON ARTIFICIAL NEURAL NETWORK

(71) Applicant: SAMSUNG ELETRÔNICA DA AMAZÔNIA LTDA., Campinas - São Paulo (BR)

(72) Inventors: Antonio Joia Neto, Campinas - São Paulo (BR); Felipe Marinho Tavares, Campinas - São Paulo (BR); Paulo Augusto Alves Luz Viana, Campinas - São Paulo (BR); Vitor Fernando Da Silva Alquati, Campinas - São Paulo (BR); Matheus De Souza Ataide, Campinas - São Paulo (BR); Lin Tzy Li, Campinas - São Paulo (BR); Daniel Eiji Higa, Campinas - São Paulo (BR); Otávio A.B. Penatti, Campinas - São Paulo (BR)

(73) Assignee: SAMSUNG ELETRÔNICA DA AMAZÔNIA LTDA., Campinas - São Paulo (BR)

(21) Appl. No.: 17/202,537

(22) Filed: Mar. 16, 2021

(30) Foreign Application Priority Data
Feb. 5, 2021 (BR) 10 2021 002255 8

(51) Int. Cl. A61B 5/00

(52) U.S. Cl. CPC A61B 5/4806; A61B 2562/0219; A61B 5/6801; A61B 5/7264

(57) ABSTRACT

An improved sleep onset/offset detection method based on a compact neural network that runs in a wearable device processing sensor data in near real-time, which means accumulating data from a few minutes instead of seconds before starting predictions.

### CROSS-REFERENCE TO RELATED APPLICATION(S)

This application is based on and claims priority under 35 U.S.C. § 119 to Brazilian Patent Application No. BR 10 2021 002255 8, filed on Feb. 5, 2021, in the Brazilian Intellectual Property Office, the disclosure of which is incorporated by reference herein in its entirety.

### TECHNICAL FIELD

The present invention relates to a method for near real-time sleep detection based on an artificial neural network running on a wearable device.

This is a very important feature for current wearable devices, as sleep detection triggers many wearable device functions, including deactivating sensors and features to save battery life, activating sleep monitoring features, and others.

When users are inactive some sensors are turned off to extend battery life, but other sensors continue active enabling methods to describe sleep sessions and providing information on when those started/ended, identifying sleep stages/events, and hence helping to infer sleep quality metrics.

An efficient solution that detects when the user is awake or not extends beyond classifying sleep stages. In the context of health and wellness, a better sleep session detection can be used to enable other technologies and solutions to improve user's quality of life.

### BACKGROUND

Commercially available wearable devices increasingly have more embedded sensors and methods that can provide users insights regarding aspects of their well-being, during sleep or active time. Those sensors are even able to assist the user to seek professional help if something abnormal is detected.

Many wearable devices in the market already provide sleep detection solutions. However, users may not have great experiences due to the occurrence of false sleep detections. Most incorrect detections occur when a user is awake watching movies or reading a book, but the method infers it as sleeping.

Some existing approaches automatically distinguish sleep and wake in time epochs based on wrist activity (actigraph) by applying a linear model whose parameters were optimized iteratively. An epoch represents k-seconds windows of data at a given sampling rate.

The use of wrist worn devices for sleep classification has been a research topic for a few decades. Common approaches can be divided into traditional methods, machine learning methods and deep learning methods, and most of them make use of activity count derived from actigraphy. Since old actigraph sensors did not have the memory capacity of modern accelerometers, the activity measures (also named activity counts) used were zero-crossing, time above threshold and digital integration, which do not require so much memory to be stored as the raw acceleration signal does.

Traditional methods are usually based on linear equations with activity counts of current, past and future epochs weighted and added. This result is then compared to a threshold to determine whether the current epoch is an asleep or awake epoch. Some other methods are based on classical machine learning techniques, such as linear regression, support vector machines (SVM) and random forests. These methods require the calculation of features to serve as input for the method. Some features used in these methods are activity count and statistics of the signal such as mean, median, and standard deviation calculated on a specific window of epochs. Deep learning approaches usually do not require features being computed to be used as input. Because of their capacity to learn representations, it is generally better to use a segment of the raw signal as input instead of hand-crafted features.

All these methods make use of the specialist-labeled data from PSG as ground truth for training/evaluating the proposed models. One such traditional method is using actigraphy data collected from subjects while they were submitted to polysomnography (PSG). The data is then used to optimize the parameters of a model of the form:

D=P(W₋₄A₋₄+W₋₃A₋₃+W₋₂A₋₂+W₋₁A₋₁+W₀A₀+W₊₁A₊₁+W₊₂A₊₂)

Epochs with D<1 are classified as sleep and D>=1 as wake, P is a scale factor, W₀, W₋₁, W₊₁ are weighting factors for the present, previous and following minutes, respectively, and A₀, A₋₁, A₊₁ are the activity scores for the present, past and following minutes, respectively. The “activity score” feature used in the sleep detection domain is as a number that represents the level of activity/movement of the user in a time period.

The article “Sleep stage prediction with raw acceleration and photoplethysmography heart rate data derived from a consumer wearable device”, published on Dec. 24, 2019, by Olivia Walch, used a dataset with 39 subjects that were submitted to PSG while wearing a wearable device for collecting acceleration data and heart rate data. They then used motion features derived from acceleration data, heart rate and an estimation of the circadian phase as features for training classical machine learning approaches like logistic regression, k-nearest neighbors, random forest and neural network.

However, in the present invention, instead of correctly detecting sleep/wake patterns during the night, the objective is to tell exactly when a person started sleeping (sleep onset) and when they woke up (sleep offset) while avoiding detecting sleep during other low-movement activities such as reading a book and watching TV. For this kind of problem, labels from PSG are not as useful since they contain little or no data prior to sleep or after waking up. Besides, the memory limitations of the device where the method will be deployed makes it hard for using approaches like deep learning. For this reason, it is used a neural network that is capable of running inference in parts between its layers, such as to allocate low memory on each epoch, and also considering information from previous epochs.

The article “Automated detection of sleep-boundary times using wrist-worn accelerometry”, published on Nov. 28, 2017, by Johanna O'Donnell, used data similar to the present invention, i.e., data collected from a free-living protocol where subjects were instructed to annotate the time they went to bed and the time they woke up. Then, this data was used to validate three different models: (1) a statistical technique for detecting change points in acceleration data series; (2) a data-driven thresholding method; and (3) a random forest. Features derived from acceleration data were used and the random forest was trained to classify whether each one-minute epoch was an asleep or awake epoch. After the classification, a rolling mean filter was used to reduce the number of erroneous wake classifications during sleep.

However, the present invention differs from the approaches (1) and (2) proposed by O'Donnell's et al. because they are not based on machine learning methods. The differences from approach (3), the random forest, are due to mainly two aspects:

The present invention used a compact neural network that considers temporal information from various minutes previous from a given time, while O'Donnell's et al. used a random forest that receives as input features extracted over acceleration data across one-minute epochs;

In the way the detection of the sleep session after sleep-wake classification is done, the present invention uses a post-processing stage based on rolling means of the model outputs and subsequent sum of recent and consecutive rolling means, of which the resulting value for each epoch is compared to thresholds in an algorithm with states for onset and offset event detection. While O'Donnell et al. used a rolling mean filter and subsequent identification of the largest block with consecutive sleep predictions to consider such predictions as onset and offset events.

The patent document CN110710962A, entitled “Sleep state detection method and device”, published on Nov. 8, 2019, by BEIJING CALORIE INFORMATION TECH CO LTD, has a work close to the present invention by proposing the use of acceleration and heart rate signals to obtain derived features/characteristics to predict sleep start, sleep end, and classify sleep stages in deep or light. CN110710962A proposed method operates as following: first it is detected if the user is wearing the device, and if that is the case, then predictions by the method can be calculated. Features are extracted from heart rate signal and acceleration signal according to an extraction window of preset duration. Heart rate change rate characteristics include, but are not limited to, the rise/fall trend of the heart rate value within a fixed period, the length of the change interval, and the jump amplitude. Acceleration data are converted into a limited number of discrete features, which include, but are not limited to, intensity of activity, duration of activity, duration of inactivity, and number of active and inactive switching.

Then, a detection method with logical conditions receives as input the extracted features to detect events of sleep start (onset) and sleep end (offset). Such detection method has a structure that includes, but is not limited to, a decision tree model, a random forest model, a support-vector-machine model, a neural-network model, etc.

Sleep staging detection is then conducted to determine stages of sleep (deep or light) based on the amount of activity and the change in heart rate during sleep. Such sleep staging detection is described by the use of thresholds applied to heart rate values, period of activity, and adjusted by prior values that can be obtained, but not limited to, manually collected data and empirical data.

The present invention, in contrast to CN110710962A, focus on minimizing predictions of false sleep sessions to provide a better user experience, and attend embedding restrictions of the solution in devices with low computational resources by using less signals and memory due the compact neural-network design.

### SUMMARY

The present invention discloses an improved sleep onset/offset detection method based on a compact neural network that runs in a wearable device, besides processing sensor data in near real-time, which means waiting to accumulate data from a few minutes instead of seconds before starting predictions.

The neural network is considered compact by having a pipeline architecture that calculates neurons values in intermediary layers (feedforward outputs) and reuse those values in future predictions, by that reducing resource usage by not processing all the ANN values for each epoch.

In order to keep the low energy consumption rate, only acceleration data was used, given that users tend to turn off light-based sensors like photoplethysmography (PPG). Given the size restriction, state of the art machine learning methods such as Deep Learning could not be applied (require much more memory/processing power). Thus, the present invention relies on an Artificial Neural Network (ANN) trained/validated/tested with a varied dataset of wearable device sensor data collected from more than 600 subjects with varied demographic characteristics.

The used datasets account for data from subjects in different free living (FL) activities (besides sleeping), and subjects that were also monitored via polysomnography (PSG) in a sleep center (SC) while also wearing a wearable device on their arm along with the whole PSG sensors attached to their body.

The present invention correctly recognizes sleep sessions and reduces greatly the false sleep session rate in comparison with the prior art proposals.

Moreover, the problem tackled herein is to identify the sleep session of a given user, defined when sleep starts (onset) and ends (offset), to avoid false sleep sessions. The data is processed by each time epoch, which in the present invention is organized as 60-seconds windows of data at 10 Hz sampling rate, leading to 600 data readings at a given time t.

Considering the mentioned restrictions, the solution was designed based on the ANN and using two different activation functions, such as Leaky ReLU and sigmoid. Feedforward outputs are also stored from many different epochs in “hidden-layers”, thus having data resulted from previous epochs in a same “hidden layer”. The goal was to have information from many previous epochs influencing the ANN output at the current epoch while also storing a small ANN data structure in memory.

Therefore, the present invention consists in a technique that detects the sleep session of a person using wearable devices with memory restrictions. Sleep session is defined as the time window that lasts between the beginning (sleep onset) and the end of sleep (sleep offset). The method was designed to run on a wearable device with memory restrictions. Specifically, given a set of readings of acceleration data, the proposed technique is capable of estimating the sleep session, showing the time at which the user slept and woke up.

### BRIEF DESCRIPTION OF THE DRAWINGS

The objectives and advantages of the current invention will become clearer through the following detailed description of the example and non-limitative drawings presented at the end of this document:

FIG. 1 presents an overview of the proposed solution.

FIG. 2 depicts the proposed ANN and its operations.

FIG. 3 illustrates the expansion of the feature extraction module.

FIG. 4 illustrates the ANN and its architecture in memory.

FIG. 5 depicts details of the final step of the post-processing module with the threshold processing by a state algorithm.

### DETAILED DESCRIPTION

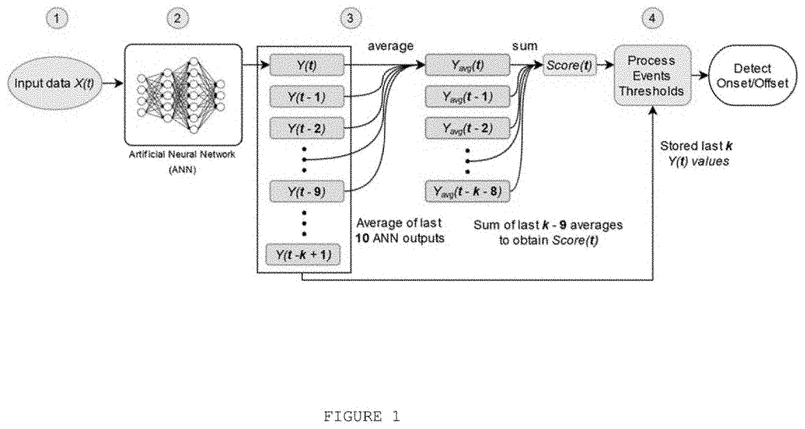

FIG. 1 depicts an overview of the proposed solution, composed of: (1) the feature extraction module that produces feature vectors to the (2) compact ANN, which outputs a prediction value for each input signal epoch. From (3) to (4) it is shown the post-ANN processing module: the ANN's outputs are accumulated in an array, averaged per epoch, and summed to yield a Score (t)—that compared with thresholds will indicate if, for the current data (epoch t), a start (onset) or end (offset) of the sleep session was identified.

The first aspect of the present invention is a neural-network pipeline architecture with optimizations that reduces the memory usage of a common feedforward inference, at the same time that can combine and make use of long-term temporal acceleration data. The present solution processes more temporal information than prior techniques, which in majority use a threshold applied to a weighted sum of previous epochs activity counts, while also keeping low memory usage for a neural-network implementation, enabling the embedding in wearable devices.

A second aspect is a post processing step, from (3) to (4), that: uses the rolling window averages of the ANN's outputs to predict sleep onset and offset by considering up to 50 minutes of previous temporal information.

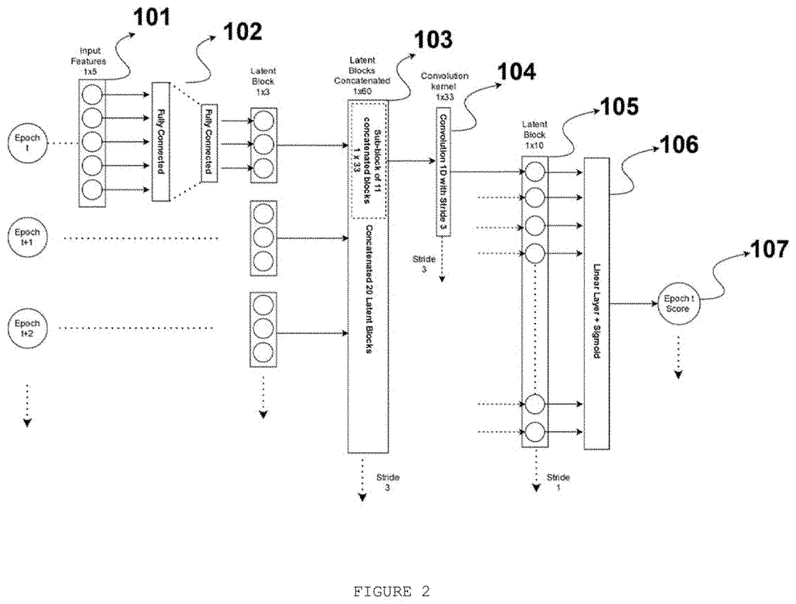

FIG. 2 details the proposed method for neural-network architecture. In this sense, the present invention uses 3-axis accelerometer measures as raw input data. For each epoch of the method, 60 seconds of data at 10 hertz is collected, totalizing 3 (axis)*60 (seconds)*10 (hertz)=1800 raw values. For each prediction, the method needs 20 minutes of data (concatenates 20 epochs). The use of the three-axis data is reduced to only the norm of the three accelerometers, wherein the norm represents the level of activity of the user accumulating all axis into one variable, reducing the abstractions the network would need to perform if three axis were used and reduces by three times the number of raw input values (from 1800 to 600).

Extracting manual features is unusual in deep learning state of the art, since most people assume that the neural network will learn the best features automatically, but to provide a memory efficient architecture, the present invention uses manually designed features so that the network's learning load is reduced, hence lowering the number of layers and neurons.

In the last step of the feature extraction (before handing it in to ANN), the 600 accelerometer-norm values are summarized into 5 manually designed features (101) and that are calculated in real-time, iteratively, at each epoch. Before passing the data through the convolution layers, the dimensionality of 5 features is reduced by using two fully connected layers (102). Those layers work as an encoder that reduces the dimensionality of the input into a latent block with 3 dimensions. Consequently, reducing the dimension from 5 to 3 results in a reduction of forty percent (40%) of the memory needed to store intermediate latent values of the network, allowing the increase of the number of epochs taken into account on the input to make one single prediction, while maintaining a low memory usage.

Twenty blocks of the latent representation of the extracted features (103) are concatenated to combine long-term temporal information from the previous calculations. Then, a convolution kernel (104) is applied in order to extract temporal information from the features. The convolution is composed by one-dimensional kernel of size K=33 and stride S=3. The output being composed of:

$${y}_{i} = {\sum }_{j = 1}^{K} {x}_{( i * S - j )} {w}_{j} + b$$

Where w∈ [symbol] ³³ are the weights of the kernel (104), x∈ [symbol] ⁶⁰ is the concatenated block of latent features (103), y∈ [symbol] ¹⁰ is the output of the convolution (105), and b∈ [symbol] ¹ is the bias of the kernel.

The convolution that uses data calculated from previous epochs is named temporal convolution, as it enables that, in deployment, the inference be done while reutilizing data calculated in previous epochs inside latent layers in the artificial neural network.

The final part of the ANN is a linear layer followed by a sigmoid function (106), combining all the convolution output to generate the score (107) for the epoch.

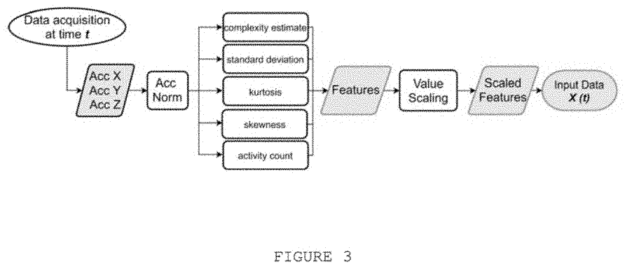

FIG. 3 illustrates the details of the feature extraction module (1). In this sense, input data are acceleration data readings for one epoch, which is one minute of data. The norm for the tri-axial acceleration raw data is obtained, from which it is calculated statistical features, such as standard deviation, skewness, kurtosis; and temporal features, such as complexity estimate and activity count.

Standard deviation, skewness and kurtosis are well established statistical measures that carry information about the signal distribution. Complexity estimate is based on the physical intuition of “stretching” a time series until it becomes a straight line. It is obtained by accumulating the variation from the value of one epoch to the next. Activity count computes how many sign changes appear in the signal value, which is also known as zero-crossing.

The feature calculations are shown in the table below, where w is the index for the w-th window, [aₓ_{_{w}}, a_{y}_{_{w}}, a_{z}_{_{w}}] are the w-th window array of the acceleration data x, y, z axis, respectively, ∥a_{w}∥ stands for the norm of the three axis of the w-th window, σ(ν) is the standard deviation of all samples in array ν, ν̅ is the mean value of array ν and Ī(C) is a function that is equal to 0 if condition C is true, and equal to 1 otherwise.

| Feature | Equation |
|---|---|
| Activity count | Σ_{i = 2}^{W} Ī(sgn(\|\|a_{w}[i]\|\| − 9.8) = sgn(\|\|a_{w}[i − 1]\|\| − 9.8)) |
| Complexity estimate | Σ_{i = 2}^{W} \|\|\|a_{w}[i]\|\| − \|\|a_{w}[i − 1]\|\|\| |
| Kurtosis | $\frac{1}{W} {\sum }_{i = 1}^{W} {( \frac{‖ {a}_{w} [ i ] ‖ - \overline{‖ {a}_{w} ‖}}{σ ( ‖ {a}_{w} ‖ )} )}^{4} - 3$ |
| Skewness | $\frac{1}{W} {\sum }_{i = 1}^{W} {( \frac{‖ {a}_{w} [ i ] ‖ - \overline{‖ {a}_{w} ‖}}{σ ( ‖ {a}_{w} ‖ )} )}^{3}$ |
| Standard deviation | $\sqrt{\frac{1}{W} {\sum }_{i = 1}^{W} {( ‖ {a}_{w} [ i ] ‖ - \overline{‖ {a}_{w} ‖} )}^{2}}$ |

In the proposed ANN, the use of Leaky ReLU over ReLU is because it does not discard negative values, even considering that they are multiplied by a very small scalar, while ReLU transforms all negative values to zero. However, better results were obtained when using Leaky ReLU in conjunction with the new method for sleep detection, though. The sigmoid function is used to concentrate the ANN's outputs in a range between zero and one. The use of the described activation functions and other ANN parameters are not intended to limit the disclosure of the invention but to exemplify its configuration in practical terms.

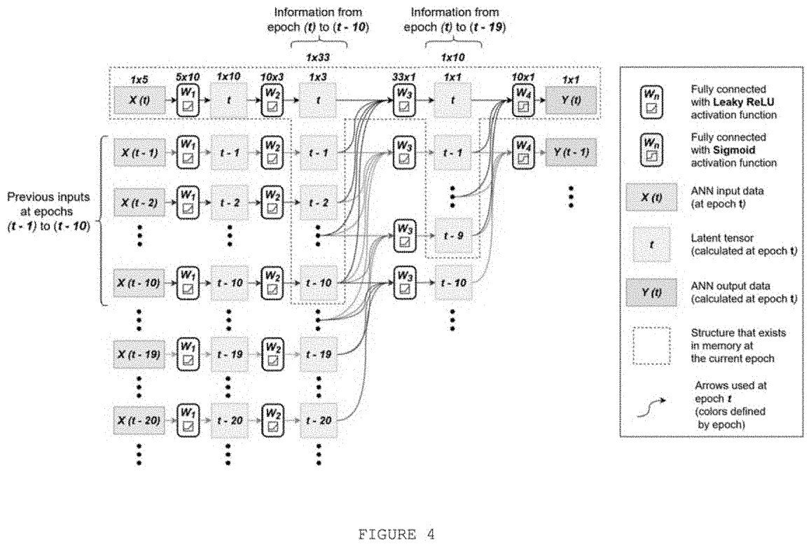

FIG. 4 details the ANN architecture with a deployment-focused perspective, addressing data in the latent tensors by the epoch it was obtained on. In this ANN representation, fully connected operations applied to epoch's data are equivalent to the FIG. 2 convolution kernels functions due the way data is represented, the resulting ANN is the same because the block with convolution stride 3 is replaced by the representation of the latent tensor with dimension 1×3. The W₃ fully connected block with Leaky ReLU also represents the convolution with kernel size 33 and stride 3, such as the W₄ fully connected block with Sigmoid also represents the convolution, but with kernel size 10 and stride 1.

In FIG. 4 layers are identified by the fully connected operations with activation functions blocks applied in them. For example, W₄ identifies both the layer of size 1×10 used in the W₄ operation and the W₄ operation itself, fully connected with the Leaky ReLU activation function.

In the FIG. 4, the rectangle in dotted line shows the ANN's structure that exists in memory on one given epoch. It is possible to identify that even by using information from 20 epochs, it is not necessary to store all the structures that would process the data for those epochs due to intermediary products of previous operations being stored in latent layers. By using this pipeline architecture, a considerably small quantity of data can be stored, in contrast to the obvious strategy of loading the entire model in memory, while also considering a good quantity of temporal information from previous epochs.

In training, the entire model represented in FIG. 2 can be allocated in memory, but for inference in deployable wearable devices the convolutional strides (104) are stored and processed individually at each epoch to reduce memory allocation. Tensors have labels indicating when their resulting values were calculated. At the present epoch (t) of processing, only the tensors with the label t are calculated.

Due the use of the disclosed temporal convolution operation, in deployment, once data is calculated for an epoch, it is not calculated again in future epochs; instead, the data inside latent layers are reutilized until they are not needed anymore. In practice, before the calculations for the next epoch begins, values are shifted inside the two latent array blocks (in FIGS. 2, 103 and 105; in FIG. 4, W₃: with information from t to t−10 and W₄: with information from t to t−19); in the first array block, the shift has stride 3 due to the dimensions of the latent tensors being 1×3, while in the second array block, the shift has stride 1 as the latent tensors have dimension 1×1.

The features X(t) are only allocated in the epoch t. The layers after W₁ and W₂ store results of dimensionality reduction. The layers after W₃ and W₄ store results from convolutions using information from previous epochs, wherein W₃ uses information from t to t−10 and W₄ uses information from t to t−19.

The convolutions W₃ are responsible for prioritizing which temporal data is important from previous calculations and are responsible for the memory usage optimization of the ANN implementation, as features X(t) and the results of the layer after W₁ are not kept in memory. All calculations, except those to obtain a value marked by the epoch t, are not calculated at the current epoch and instead are buffered, since epoch t−n when it was calculated, and kept in memory in layers after W₂ and W₃, which are shifted from the buffer until exiting the layer.

As illustrated in FIG. 1, the post-processing step (3) uses ANN outputs to detect a sleep onset or offset based on certain conditions. The means of Y(t) to Y(t−9) outputs of the ANN are averaged to calculate Y_{avg}(t), then the last k−9 most recent values of Y_{avg} are summed resulting in a Score(t). Score(t) values range from 0 to k−9, in which low values indicate the start of a sleep session, while high values indicate its end.

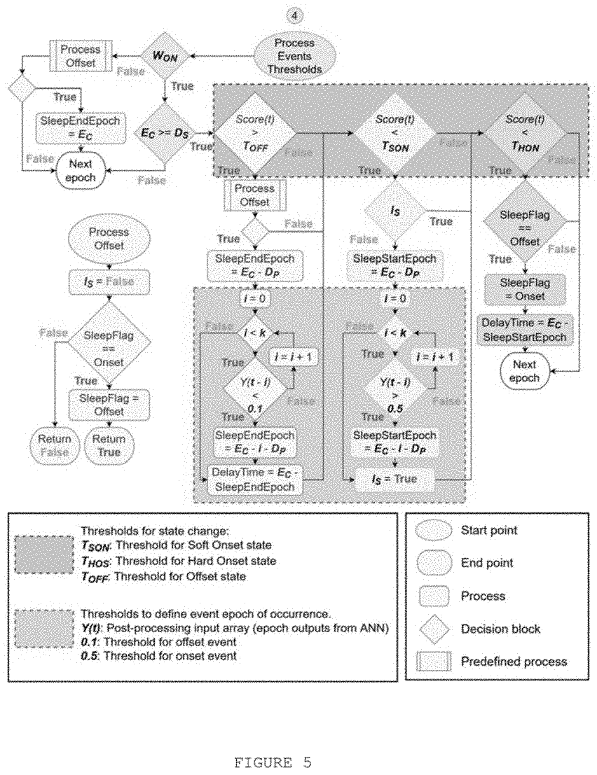

FIG. 5 details the final step of the post-processing, which is a state machine that changes states based on threshold condition values. Its input is an array of ANN outputs, where the i-th element was the ANN output in epoch t−i (where i=0 is the current epoch). For this invention implementation example, the input array has size 31, thus 31 minutes of ANN's outputs are used. Moreover, thresholds are defined for the quantities of accumulated ANN outputs (k), number of average sums (10), Score(t) thresholds, and Y(t) thresholds. Those were chosen by design and by parameter search during the training/validation phase of the present invention.

Therefore, the post-processing module only detects new onset/offset events if both following conditions are true: enough epochs elapsed since the post-processing started (Dₛ, “Device Started”), and if, in the last epoch, other algorithms in the wearable device indicate that the user is still wearing the device (W_{ON}, “Wearable On”).

The post-processing state machine has three states referring to sleep event detection thresholds: soft onset, hard onset, and offset. The soft onset state does not trigger the onset event in the algorithm output, but it is used to store when the onset event might have occurred if the next state transition is the hard onset state. The hard onset state confirms that an onset event occurred and triggers the signal that detected this event using the stored epoch at the soft onset state to indicate in which epoch the onset happened. The offset state triggers the offset event and indicates when the offset happened.

The thresholds T_{HON} (Hard Onset Threshold) and T_{OFF} (Offset Threshold) determine, respectively, an onset or offset event when compared with Score(t). The trigger of the T_{SON} (Soft Onset Threshold) indicates the epoch an onset event occurs, if T_{HON} is obtained before T_{OFF}. Then, if a T_{HON} threshold is reached, the candidate onset epoch is the one when T_{SON} happened, so this state serves as a memory. To better indicate when onset and offset events occurred, the last k Y(t) values of a T_{SON} or T_{OFF} event are searched, and the epoch t with Y(t)>0.5 is defined as the onset epoch (in the case of T_{SON}), or with Y(t)<0.1 is set as the offset epoch (in the case of T_{OFF}) .

A number of epochs (D_{P}) is subtracted in every event detection to better indicate at which epoch that event happened. Auxiliary variables are also used for counting epochs (E_{C}, “Epoch Count”), and keeping track of the state between soft onset and hard onset (I_{S}, “Is Soft”).

The proposed method uses information from 50 minutes (50 epochs) to make a sleep onset or sleep offset prediction. This can be verified by: k values of previous ANN outputs (4), as k=31 and the k's 31th value were obtained in the ANN by considering information from its previous 19 minutes (19 epochs), as shown in FIG. 4, in total 31+19=50 minutes of temporal information is used for a prediction. At any time, the present invention stores four variables that can be consulted by external services: i) SleepFlag indicating if the latest sleep session event was an onset or offset, ii) DelayTime storing how many epochs ago the latest event occurred, iii) SleepStartEpoch with value for which epoch registered the latest onset event, and iv) SleepEndEpoch with value for which epoch registered the latest offset event.

To select the best model and parameters for the solution, an end-to-end evaluation is conducted considering results from the ANN model training and the post-processing parameter grid search.

During training and validation, features are calculated using the following procedure. Firstly, the 3-axial acceleration data is used to calculate the acceleration data norm. Then derived features are calculated using segments of W seconds, this segment slides over the signal with a defined stride S. The i-th segment used for feature calculation is the window from time t=i*SR*S to t=(i*SR*S)+W, where SR is the sampling rate of the signal. Five features are calculated for each segment. These features are repeated N times, so the feature vector will have 5*N features, each consecutive repetition is delayed from the previous by 1 epoch. This is done because the model needs features from N=20 segments. For the training dataset S=30 and for the validation dataset (and inference operation) S=60 and W=120.

The values for variables, parameters, and thresholds described in this invention are the ones found after one execution of the technique training/validation procedure. These numbers are not restrictive for the invention, and, depending on the training dataset and stochastic training behavior, different values can and possibly will be found from the ones stated in this detailed description.

The ANN's weights are initialized using a normal distribution ND(0,std²), where std is the standard deviation and the biases are also randomly initialized using a normal distribution ND(0,1). The weights were updated during the training step using batches of size 256 to calculate the gradients and, as the weights were being updated, the model was being evaluated in the validation data using the cohen kappa score metric. If the model achieved a new higher cohen kappa, the model weights were saved. If the model trained for 20 epochs without reaching a better cohen kappa score or reached a total of 1000 training epochs, the training is stopped.

The training is halted to prevent the model to continue a training where the parameters already overfitted. The Rectified Adam (RAdam) technique was used as the optimizer to update the weights during the training. RAdam is more robust than the classic Adam algorithm, being almost invariant to the initial learning rate due to its weight updating policies. The loss function for training is the binary cross-entropy.

Due to the inherent stochastic nature of the neural network, a certain amount of training was conducted varying the seed for weights initialization. To reach the results presented here, a total of 39 ANNs of the same proposed architecture, but with different initial weights, were created and trained using the same scheme as described above.

The present solution uses a post-ANN processing module that has 5 parameters, so it is not sufficient to use the best model of the ANN in the validation set regarding the loss value nor the cohen kappa score, because the post-processing module, which comes after the ANN to trigger or not sleep session events in the end. So, a grid-search is applied with all trained neural networks to find the best combination of ANN weights and post-processing parameters. The grid search used for the presented results is:

i. Varying k from 21 to 46, in steps of 5.

ii. Varying D_{P} from 0 to 8, in steps of 2.

iii. Varying T_{SON} from 1 to the minimum between 16 and k−9 (maximum value Score(t) can reach), in steps of 3.

iv. Varying T_{HON} from 0.25 to 4, varying by a factor of 2 (at each step the value is multiplied by 2).

v. Varying T_{OFF} from (k÷4)+4 to the minimum between 40 and k−9, in steps of 2.

For evaluation purposes, sleep sessions that are smaller than 1 hour are ignored since methods in higher abstraction levels can easily ignore them. For the evaluation metrics, the following definitions are considered:

i. Recording is a set of sensor data recorded continuously by wearable devices.

ii. Subjects are people that had data collected by wearable devices. A subject in a dataset can have one or more recordings.

iii. Ground Truth (GT) or Golden Standard are annotated by specialist as the correct answer (for sleep session, start and end of the sleep, wake/sleep epoch, etc.);

iv. Sleep Session (SS) is the segment in a recording with start and end epoch of a sleep session;

v. Ground Truth Sleep Session (GS) is the golden standard Sleep Session;

vi. Predicted Sleep Session (PS) is the sleep session detected or predicted by a method;

vii. No Predicted Sleep Session (NS) is the case that a method did not detect sleep session for a recording file. This does not evaluate success or errors.

For each combination of model weights and parameters, the following metrics are calculated for evaluation purposes: total offset error (sum of all offset errors), total onset error (sum of all onset errors), number of cut sleep sessions, number of missed sessions, number of false sessions, and intersection over union. Their descriptions are as follow:

(i) False sleep sessions are those that method predicted as sleep sessions, but user was actually awake during the entire session. In the results, the percentage of cases the method went wrong on its sleep session predictions;

(ii) Average sleep onset error indicates, in number of epochs, the average difference between predicted and GT sleep start, in the evaluation/test dataset.

(iii) Average sleep offset error indicates the average number of epochs difference between predicted and GT sleep end, in the evaluation/test dataset.

(iv) Cut sessions count how many times the method predicted interruptions in the sleep session, like two or more sleep sessions with a “wake session” between them (representing cuts), instead of only one longer session as expected by GS.

(v) Missed sleep sessions are those sleep sessions that are in the dataset, but the method did not detect;

(vi) Intersection over Union (IoU) for Sleep Session provides the measure of how much the PS fits its GS and it is summarized by IoU=(PS∩GS)÷(PS∪GS), where perfect fits are equal to 1 and no intersections is 0;

(vii) Correctly predicted sessions are the proportion of the recordings in dataset that method predicted correctly that there is sleep or no sleep sessions in the recordings, that is: (PS_{correct}+NS_{correct})÷(Total of Recordings)

The limits for each parameter in the grid search are chosen by looking at how the method works, for instance: T_{OFF} needs to be at most k−9, and at least T_{SON} for the model to work properly, and T_{HON} needed to be at least 0 and at most T_{SON}. This makes these parameters bounded by k, which was chosen based on how much memory could be used, since it dictates the size of the buffer vector that stores past scores. The parameter D_{P} is independent, and the upper limit is chosen empirically, when verifying the maximum value at which this parameter yields good metrics. The limits for second grid search are chosen by looking at the results of the first one and analyzing the lower and upper bounds at which each parameter would yield good metrics.

The process to filter and choose the overall best candidates is done by inspecting results in term of multiple evaluation metrics in train data and validation data splits.

Moreover, at least one of the plurality of modules may be implemented through an AI model in the present invention. A function associated with AI may be performed through the non-volatile memory, the volatile memory, and the processor.

The processor may include one or a plurality of processors. At this time, one or a plurality of processors may be a general-purpose processor, such as a central processing unit (CPU), an application processor (AP), or the like, a graphics-only processing unit such as a graphics processing unit (GPU), a visual processing unit (VPU), and/or an AI-dedicated processor such as a neural processing unit (NPU).

The one or a plurality of processors control the processing of the input data in accordance with a predefined operating rule or artificial intelligence (AI) model stored in the non-volatile memory and the volatile memory. The predefined operating rule or artificial intelligence model is provided through training or learning.

Here, being provided through learning means that, by applying a learning algorithm to a plurality of learning data, a predefined operating rule or AI model of a desired characteristic is made. The learning may be performed in a device itself in which AI is performed, according to an embodiment, and/or may be implemented through a separate server/system.

The AI model may consist of a plurality of neural network layers. Each layer has a plurality of weight values and performs a layer operation through calculation of a previous layer and an operation of a plurality of weights. Examples of neural networks include, but are not limited to, convolutional neural network (CNN), deep neural network (DNN), recurrent neural network (RNN), restricted Boltzmann Machine (RBM), deep belief network (DBN), bidirectional recurrent deep neural network (BRDNN), generative adversarial networks (GAN), and deep Q-networks.

The learning algorithm is a method for training a predetermined target device (for example, a robot) using a plurality of learning data to cause, allow, or control the target device to make a determination or prediction. Examples of learning algorithms include, but are not limited to, supervised learning, unsupervised learning, semi-supervised learning, or reinforcement learning.

Although the present invention has been described in connection with certain preferred embodiments, it should be understood that it is not intended to limit the disclosure to those particular embodiments. Rather, it is intended to cover all alternatives, modifications and equivalents possible within the spirit and scope of the disclosure as defined by the appended claims.

What is claimed is:

1. A method of near real-time sleep detection in a wearable device based on artificial neural network, comprising:
receiving an input signal from an accelerometer;
extracting input data X(t) from raw data provided by the accelerometer;
producing a feature vector from extracted features;
inputting the feature vector in the Artificial Neural Network (ANN);
applying a convolution kernel as part of the ANN to extract temporal information of the features;
accumulating previous temporal information in latent ANN layers;
applying a linear layer followed by a sigmoid function, combining all convolution output;
generating the output averaged array of the ANN from t to t−9;
generating the Score(t) by summing the last k−9 averaged arrays;
establishing processing events thresholds; and
post-processing an array of ANN outputs in a state machine, determining the state of a user by a current epoch.

2. The method as in claim 1, wherein the input signal comprises tri-axial acceleration data readings for one epoch.

3. The method as in claim 2, wherein the tri-axial acceleration data is reduced to its norm over three axes.

4. The method as in claim 1, wherein the extraction of input data X(t) is further summarized into 5 features calculated iteratively comprising:
statistical features comprising standard deviation, skewness, and kurtosis; and
temporal features comprising complexity estimate and activity count.

5. The method as in claim 1, wherein the dimensionality of 5 features is reduced to a latent block with 3 dimensions by using two fully connected layers W₁, W₂.

6. The method as in claim 1, wherein 20 latent blocks of the extracted features are concatenated, combining long-term temporal information from previous calculations.

7. The method as in claim 1, wherein a convolution kernel is applied to extract information from the concatenated latent blocks, wherein the convolution is composed by one-dimensional kernel of size K=33.

8. The method as in claim 1, wherein the output of the convolution kernel comprises:
$${y}_{i} = {\sum }_{j = 1}^{K} {x}_{( i * S - j )} {w}_{j} + b$$
where w∈ [symbol] ³³ are the weights of the convolution kernel, x∈ [symbol] ⁶⁰ is the concatenated block of latent features, y∈ [symbol] ¹⁰ is the output of the convolution, and b∈ [symbol] ¹ is the bias of the kernel.

9. The method as in claim 1, wherein the convolutional layers W₃ store information from t to t−10 epochs.

10. The method as in claim 1, wherein the convolutional layers W₄ store information from t to t−19 epochs.

11. The method as in claim 1, wherein the post processing presents three states for event processing: soft onset, hard onset and offset.

12. The method as in claim 1, wherein four variables to be consulted by external services are stored during the post-processing with predicted sleep session information: SleepFlag; DelayTime; SleepStartEpoch; and SleepEndEpoch.

13. The method as in claim 1, wherein a grid-search is applied with all trained neural networks to find the best combination of ANN weights and post-processing parameters.

14. The method as in claim 1, wherein the grid search comprises:
varying k from 21 to 46, in steps of 5.
varying D_{P} from 0 to 8, in steps of 2.
varying T_{SON} from 1 to the minimum between 16 and k−9 in steps of 3.
varying T_{HON} from 0.25 to 4, varying by a factor of 2.
varying T_{OFF} from (k÷4)+4 to the minimum between 40 and k−9, in steps of 2.

15. The method as in claim 1, wherein sleep sessions that are smaller than 1 hour are ignored.
